(19) United States
(12) Patent Application Publication
(10) Pub. No.: US 2007/0016011 A1
(43) Pub. Date: Jan. 18, 2007

(54) INSTRUMENT POSITION RECORDING IN MEDICAL NAVIGATION

(76) Inventors: Robert Schmidt, Munich (DE); Nils Frielinghaus, Heimstetten (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 11/419,073

(22) Filed: May 18, 2006

Related U.S. Application Data
(60) Provisional application No. 60/685,937, filed on May 31, 2005.

(30) Foreign Application Priority Data
May 18, 2005 (EP) 05010747.3

(51) Int. Cl. A61B 5/05

(52) U.S. Cl. 600/424

(57) ABSTRACT

A medical navigation apparatus and method determines, tracks and represents a position of an instrument in relation to acquired anatomical image data, and includes recording logic configured to record positions of one or more functional sections of the instrument over a period of time.

### RELATED APPLICATION DATA

This application claims priority of U.S. Provisional Application No. 60/685,937 filed on May 31, 2005, which is incorporated herein by reference in its entirety.

### FIELD OF THE INVENTION

The present invention relates to medical navigation and, more particularly, to a medical navigation device and a method for the medical navigation of an instrument.

### BACKGROUND OF THE INVENTION

Medical navigation systems provide visual support in real time by representing a previously acquired anatomical image data set together with, and registered with respect to, the current position of an instrument. Thus, the user can see a virtual representation of where an instrument tip is located in relation to the anatomical setting at any point in time, even when the user cannot see the instrument tip with the naked eye. In other words, such navigation systems assist in correlating the current position of instruments with anatomical information derived from imaging equipment (e.g., from CT, MR, x-ray equipment, etc.). An exemplary navigation device is described in DE 196 39 615 A1, which is hereby incorporated by reference in its entirety.

### SUMMARY OF THE INVENTION

A medical navigation device and method that determine, track and represent a position of an instrument in relation to acquired anatomical image data are realized by a navigation device that includes recording logic. By means of the recording logic, the positions of one or more functional sections of the instrument can be recorded over a period of time.

In other words, information relating to the whereabouts or treatment locations of surgical instruments (or their functional sections) can be made available to the user for a single point in time, multiple points in time, or periods of time, wherein the respective time period is prior to the current point in time (real-time). The navigation system can “remember” a location of the functional section of the instrument a certain period of time before the current point in time. This information can provide the user a number of new options with respect to planning and/or carrying out his treatment. These options are explained in more detail below.

In accordance with one embodiment, a navigation device can be designed such that the recording logic includes at least one of the following elements:
a data processor for generating the positional data to be recorded;
a storage medium for recording the positional data;
an image representation for displaying the positions; and
an input unit for triggering, changing or terminating the recording and/or the representation/display.

In accordance with another embodiment, there is provided a method that includes displaying the recorded positions in relation to and together with the anatomical image data on an image representation unit. Thus, the user can be shown the location where the functional part of the surgical instrument has already been. These data, however, do not change during treatment, not even when the treatment changes the setting of the anatomical data (e.g., when a fluid is siphoned or vacuumed away). A display of the positions, however, can illustrate to the user where he already has vacuumed away the fluid. Therefore, the treatment can be carried out more effectively, precisely and optimally.

Another way of realizing the method (also in combination with the aforementioned method) can be to identify the recorded positions as additional anatomical data and to add them to the acquired image data set as additional information or amended information (e.g., concerning parts of soft tissue that have been pressed in or deformed). Instruments that move within an anatomical setting and, for example, hit against walls from the inside, can supply additional data concerning the present anatomy that may not yet have been possible to capture in advance. In any event the recorded positions supply current data. This fact has up until now remained unconsidered, since, for example, attention has always been focussed on displaying the current whereabouts of the instrument tip in real time. The present invention is the first to recognize the value of information from the previous course of functional instrument sections, which includes the ability to obtain additional and current anatomical data.

These additional data can be used in the most varied of ways. Using the additional data, for example, the anatomy can be registered or re-registered with respect to the image data set, e.g., the navigation system can be informed of the position correlation between the anatomy actually present and the anatomy available in the computer as a data set. Furthermore, the additional anatomical data can be used to document changes in the anatomy over a longer period of time. The additional anatomical data also can serve as a verifying tool, wherein either the registration and/or the contents of the image data set can be verified. The recorded positions can be used to analyze or verify actions taken on the anatomy.

The invention further provides a program which, when running on a computer or loaded onto a computer, causes the computer to carry out a method such has been explained above. The invention further provides a computer program storage medium comprising such a program.

### BRIEF DESCRIPTION OF THE DRAWINGS

The forgoing and other embodiments of the invention are hereinafter discussed with reference to the drawings.

FIG. 1 illustrates an instrument guided on or in a patient, wherein the instrument is tracked by a medical navigation device.

FIG. 2 illustrates an instrument in an anatomical section comprising an exemplary area of effect in accordance with the invention.

FIG. 3 illustrates an exemplary surgical instrument and the exemplary recorded positions relative to the anatomy in accordance with the invention.

### DETAILED DESCRIPTION

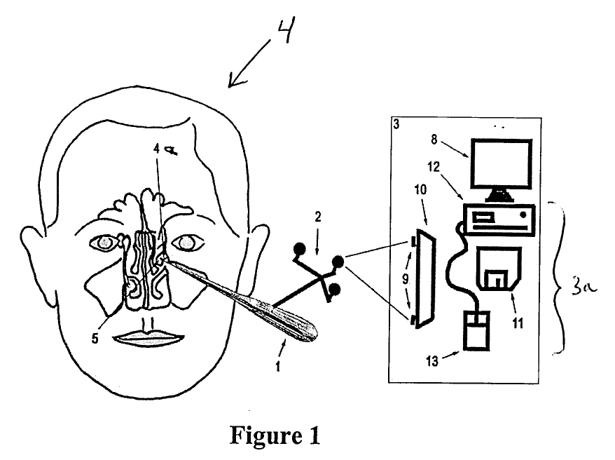

FIG. 1 shows an exemplary medical navigation system 3, an instrument 1 with a reference array 2 attached thereto, and a patient's anatomy 4 (head with frontal/accessory sinuses). The navigation system 3 includes two tracking cameras 9 on a tracking unit 10, an image display unit 8 and a navigation unit 3a. The navigation unit includes a data processor 12, a storage medium 11 and an input unit 13, as indicated in FIG. 1. The data processor 12, in preparation for display of data on the image display unit 8, processes positional data captured via the cameras 9, and the processed and/or raw positional data are recorded on the storage medium 11. Further, the data is displayed on the image display unit 8, together with other relevant data.

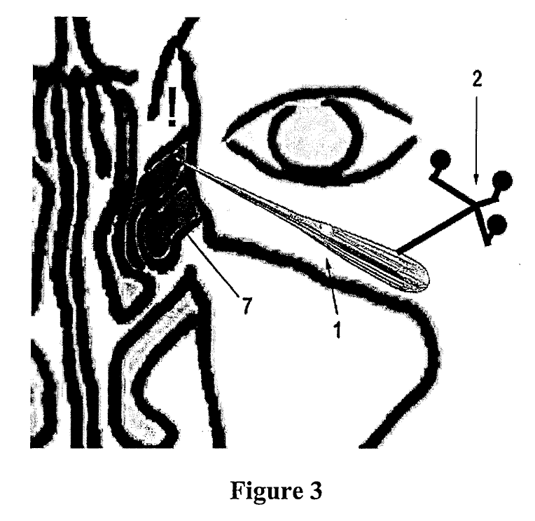

The input unit 13 is schematically represented, as are the other units, and can serve to trigger, change or terminate the recording and/or the representation/display. The input unit 13 need not be provided separately as shown in FIG. 3. For example, the image display unit 8 can be embodied as a touch-sensitive screen and therefore also as an input unit.

The patient anatomy 4, which is represented alongside the navigation system 3, is shown in detail, for example, with regard to the accessory sinuses of the nose and the frontal sinuses. The instrument 1, represented by any suitable instrument, has a functional tip 5 lying in an accessory sinus of the nose 4a. A location of the instrument 1 and/or the functional tip 5 can be determined by the navigation system 3 using the reference array 2 (which includes three reference markers). As the configuration of the instrument 1 itself also is known to the navigation system 3, the position of the tip 5 is known by means of the reference array 2. If the instrument is not “logged” in the navigation system 3, the distance of the tip 5 from the reference star 2 can be determined or input in advance.

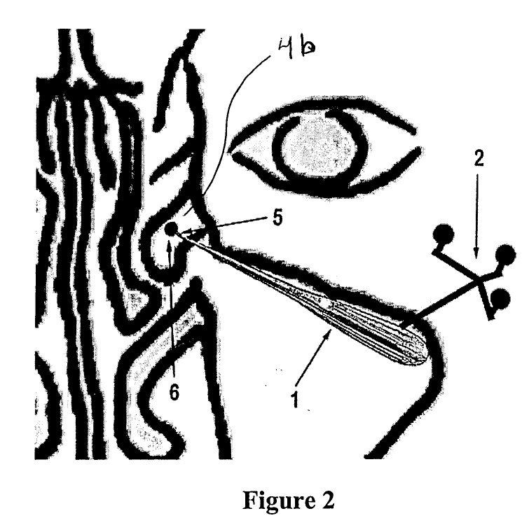

FIG. 2 shows an enlarged view of the instrument 1 and tip 5 lying in a cavity 4b. The area of effect 6 of the instrument 1 also is shown. The instrument 1 may have a vacuum or siphon device on its tip 5 to enable the removal of fluid, for example, and the area of effect 6 would then show the space being vacuumed by the instrument 1 at a particular point in time. With further reference to FIG. 3, there is shown a marked area 7 which in effect comprises an accumulation of the areas of effect 6 in FIG. 2 over a previous period of time. Here, for example, the area 7 has already been “vacuumed clean”.

The invention improves a medical navigation system by recording positions of an instrument that are observed during treatment, scanning or when the instrument is used in some other way. The recorded positions, for example via highlighting on the displayed image, are used to represent those parts of the anatomical data that the instrument has already probed or been swept over, or to modify pre-operative acquired images so that they mirror the current anatomical state (for example, by removing parts of the images or deforming the images on the basis of acquired data, which could also be referred to as “scan data”).

The invention allows the surgeon to compare the planned treatment targets with those which the surgical instrument has probed during the treatment. In the event that portions of the anatomy as shown in the planned image data are not visible (e.g., cavities, tumor portions, etc.), and the user encounters or is made aware of such areas during the course of a procedure, the user can continue with the treatment by moving the instrument to those areas which have not yet been treated/marked. This can be advantageous, for example, when treating the accessory sinuses or frontal sinuses, as it enables one to identify those sinuses that have not yet been cleaned. Further, in neurosurgery, for example, the invention enables identification of parts of a tumor which have not yet been removed.

The invention also enables visualizations to be updated to better align them with the intra-operative scenario, so that the surgeon can optimally orientate himself. This could be of use in neurosurgery, for example, when parts of the cranial bone are removed and the visualization of this “hole” in the cranium can aid the surgeon in assigning the visible information to the information in the anatomical images.

Some navigation systems are or can be integrated with planning systems that include a drawing function. The present invention can combine a surgical navigation system with the drawing function of a surgical planning system in such a way that the treatment instrument 1 is tracked and represented in the usual way, but the position of the instrument tip 5 is used to draw an object (just as a mouse is used to draw an object on a screen). The area of effect 6 would then optimally also be used, as shown in FIG. 2, as the area that draws something in or on the representation. The “brush size” can be advantageously defined depending on the instrument, wherein safety margins could be added or removed. The “brush size” could of course also be selected by the user himself.

Drawing or painting 7, as shown in FIG. 3, is performed either during the whole treatment or in predetermined verifying steps for the treatment (e.g., at the end). If the drawing 7 is inserted as a whole into the representation at the predetermined verifying steps, a specially constructed instrument could be used (e.g., one with a large, round tip which cannot cause any injuries). If drawing 7 is only to be performed at predetermined steps (e.g., representation is only intended at these particular points in time), a user input can be used to inform the system when there is to be drawing or representation. This user input can be the usual user input of the navigation system (e.g., the input unit 13 in FIG. 1).

All of these techniques of representation are possible, since the positions of the functional section of the instrument 1 or instrument tip 5 are recorded by the navigation system 3. In the verification steps for the treatment, the surgeon can see the areas that an instrument has already been (e.g., as two-dimensional or three-dimensional reconstructions of the portrayed object superimposed on the anatomical images or compared with reconstructions of the planned target area). FIG. 3 shows such a situation. The locations which have already been treated are represented as filled in or painted areas (reference sign 7), while an exclamation mark (!) can identify or mark a location that has not yet been treated.

Depending on the results, the surgeon then can decide whether to proceed with the treatment and recording (completing the object) and when a second verification step is to be carried out. It is also possible, by including the treatment measures, to visualize the current scenario and create a representation which corresponds to the actual, current treatment target location.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A medical navigation device for determining, tracking and representing a position of an instrument in relation to acquired anatomical image data, comprising a recording logic configured to record positions of one or more functional sections of the instrument over a period of time.

2. The navigation device as set forth in claim 1, wherein the recording logic comprises at least one of
a data processor for generating the positional data to be recorded;
a storage medium for recording the positional data;
an image representation for displaying the positions; or
an input unit for triggering, changing or terminating the recording and/or the representation/display.

3. A method for medical navigation of an instrument, wherein a position of the instrument in relation to acquired anatomical image data is determined, tracked and represented, comprising recording positions of one or more functional sections of the instrument over a period of time.

4. The method as set forth in claim 3, further comprising displaying the recorded positions on an image representation unit in relation to and together with the anatomical image data.

5. The method as set forth in claim 3, wherein recording includes identifying the recorded positions as additional anatomical data, and said additional anatomical data are added to the acquired image data set as additional information or amended information.

6. The method as set forth in claim 5, further comprising using the additional anatomical data to register the anatomy with respect to the image data set.

7. The method as set forth in claim 5, further comprising using the additional anatomical data to document changes in the anatomy over a period of time.

8. The method as set forth in claim 5, further comprising using the additional anatomical data to verify registration or verify the contents of the image data.

9. The method as set forth in claim 3, further comprising using the recorded positions to analyze or verify actions taken on the anatomy.

10. A computer program embodied on a computer readable medium for medical navigation of an instrument, wherein a position of the instrument in relation to acquired anatomical image data is determined, tracked and represented, comprising code that records positions of one or more functional sections of the instrument over a period of time.

11. A system for recording an instrument position, comprising:
at least two cameras for tracking the instrument, said cameras communicatively coupled to said processor circuit, and;
a recording system, comprising logic that records positions of one or more functional sections of the instrument over a period of time.

12. The system as set forth in claim 11, further comprising:
a processor circuit communicatively coupled to the recording system, said processor circuit including a processor and a memory; and
a display unit for displaying graphical images,
wherein the processor circuit generates image data based on the recorded positions of the instrument and provides said generated image data to the display unit.
